(19) United States
(12) Patent Application Publication
(10) Pub. No.: US 2008/0300677 A1
(43) Pub. Date: Dec. 4, 2008

(54) Method of treatment and devices for the treatment of left ventricular failure

(76) Inventor: Howard L. Schrayer, Princeton, NJ (US)

Correspondence Address: Kenneth Watov, Esq.;WATOV & KIPNES, P.C., P.O. Box 247, Princeton Junction, NJ, 08550, US

(21) Appl. No.: 11/975,567

(22) Filed: Oct. 19, 2007

Related U.S. Application Data
(62) Division of application No. 10/957,436, filed on Oct. 1, 2004.

(51) Int. Cl. A61F 2/24; A61B 19/00

(52) U.S. Cl. 623/2.12; 128/898; 623/2.18; 623/2.38

(57) ABSTRACT

The effects of acute left ventricular heart failure are mitigated by temporary support of the cardiac function through use of either one or both of an expendable temporary one-way valve positioned in the aorta, having a collapsible frame that is expanded upon deployment, and/or a temporary dilation device positioned in the descending aorta for expanding upon deployment to increase the diameter of the associated portion of the aorta. When used together, the dilation device is positioned distal to the temporary one-way valve.

### RELATED APPLICATION

This application is a Divisional from co-pending prior application Ser. No. 10/957,436, filed on Oct. 1, 2004.

### FIELD OF THE INVENTION

This invention relates to temporary cardiac assist devices employed to provide functional support to a diseased, traumatized or failing heart for a limited time until the heart recovers sufficiently to perform effectively without support or until a longer-term treatment is provided. In particular, the invention relates to collapsible, non-powered devices that are introduced through percutaneous transluminal techniques to decrease the resistance against which the heart must pump.

### BACKGROUND OF THE INVENTION

Acute left ventricular heart failure can occur episodically in a patient suffering from chronic congestive heart failure (CHF) or from a specific acute stress situation. Some typical stress situations include myocardial infarction, unstable angina, cardio-surgery and catheter-based coronary interventions. The condition is characterized by a reduction in cardiac output, increased left ventricular end diastolic pressure and volume, decreased pump efficiency (reduced ejection fraction) and increased after load (outflow resistance). The increase in outflow resistance may arise from several factors including hypertension, aortic stenosis and poor peripheral run-off.

The therapeutic reduction in after load (the resistance against which the heart contracts) has become an important treatment for heart failure. This has been addressed pharmacologically through the use of antihypertensive drugs and vasodilators. A few medical device systems have been developed that may manage after load as part of the cardiac assist or support function. For example, the intra-aortic balloon pump (IABP) has been used as a temporary mechanical heart assist device in episodes of acute left ventricular failure.

The IABP comprises a percutaneously introduced balloon catheter that is positioned in the aorta, and a control console that times the inflation/deflation cycle of the balloon to augment cardiac performance. The balloon is deflated during systole to reduce outflow resistance and inflated during diastole to propel blood forward and to augment coronary artery perfusion (counter-pulsation). As the heart recovers from the acute incident the patient is gradually weaned from IABP support. This may be accomplished by reducing the balloon pump volume and/or by reducing the percentage of cardiac cycles during which the IABP is activated. Although this system is widely used, it is expensive, requires careful and nearly continuous adjustment and its use requires frequent monitoring by a skilled medical technologist. The system requires that the balloon inflation/deflation cycle be electronically timed to coincide with the patient's cardiac cycle.

A number of prior mechanical device inventions have been made for the treatment of heart failure, particularly left ventricular heart failure. Nearly all of these inventions are dependent on the use of an external power source for operation; and all of the systems that support the function of the heart by augmenting pulsatile flow of blood require that the device operation be timed to coincide with some portion of the natural rhythm of the heart.

U.S. Pat. No. 4,388,919 (Benjamin) and U.S. Pat. No. 4,881,527 (Lerman) describe systems that support the circulation by external compression means of the torso or peripheral limbs. U.S. Pat. No. 6,254,525 (Levin) describes an inflatable bladder that is positioned around the heart to provide pulsatile support by compressing the heart.

U.S. Pat. No. 4,902,273 (Choy) and U.S. Pat. No. 5,176,619 (Segalowitz) describe support systems that employ intra-ventricular balloon pump means.

U.S. Pat. No. 5,800,334 (Wilk) describes a balloon support system that is positioned within the pericardial space; and U.S. Pat. No. 4,902,272 (Milder) describes an intra-aortic balloon pump device.

U.S. Pat. No. 6,193,648 (Krueger) describes a mesh jacket that is snugly positioned around the heart to prevent continued enlargement due to congestive heart failure. In theory this limits the rate of degradation of cardiac performance. The device is non-powered, does not require a timing mechanism. However, implantation of the device requires a significantly invasive surgical procedure.

Several prior art devices are directed at replacement of the diseased natural aortic valve (i.e. to treat aortic valve insufficiency). A number of these devices are directed toward percutaneous transluminal introduction of an aortic valve prosthesis that is intended to replace or supplant the function of the natural aortic valve. In order for these devices to perform their intended function the natural heart valve must be removed or rendered non-operative. None of these devices is designed with the intention of use as a temporary treatment for acute heart failure by functioning in concert with a relatively normal natural aortic valve. Also, the known prior art does not provide temporary implantable non-powered devices for the treatment of the failing left ventricle.

Previously described percutaneously introduced valve inventions are designed to fit within a specific diameter annulus or implant site depending upon the anatomic dimensions of the individual patient. A number of the prior patents that describe percutaneous transluminal introduction of an aortic valve prosthesis are described below to illustrate the existing technology and to assist in providing an understanding of the features that differentiate the present invention from the prior art.

U.S. Pat. No. 3,671,979 (Moulopoulos) describes percutaneous introduction of a prosthetic heart valve that can be repositioned and removed and is intended to replace the function of a diseased natural aortic valve. This device is inserted into the vessel in a collapsed form and is deployed like an umbrella with the apex of the umbrella (cone) pointing upstream toward the heart. This configuration provides no means for centering the valve within the aorta. In principle, the arrangement allows the valve leaflets to contact the aortic wall during diastole and thus prevent reverse flow. The design does not permit central blood flow; and the area immediately downstream and within the umbrella has no flow or low flow of blood. This design configuration can lead to clot formation and ultimately release of a dangerous clot. This patent also illustrates a percutaneous valve that is introduced as a deflated balloon. The balloon must be externally powered and requires a timing mechanism to synchronize the inflation/deflation cycle with the cardiac rhythm. This concept is also illustrated in International Publication Number WO 00/44313 (Lambrecht, et al.).

U.S. Pat. No. 4,056,854 (Boretos, et al.) describes percutaneous introduction of a prosthetic heart valve that is intended to replace the function of the natural aortic valve, but may remain tethered to an extension stem so that it can be re-positioned or removed at a later date. The valve annulus is formed by a series of springs connecting the distal ends of outwardly biased support wires. The valving mechanism is a single flexible tubular membrane that surrounds the frame formed by the annulus and the support wires. The entire valve assembly is constrained within a capsule during introduction. This design requires a large vascular access incision due to the size of the capsule and the non-compressible spring components. The design depends upon the random collapse of the tubular membrane to prevent retrograde flow.

U.S. Pat. No. 6,168,614 (Andersen et al.) and U.S. Pat. No. 5,855,601 (Bessler et al.) describe prosthetic valves that are intended as permanent implants to assume the function of the natural aortic valve. The inventions include mechanisms for fixing the structure that forms the valve annulus to an intravascular site such as the natural valve annulus after the natural valve has been removed.

It is known in the prior art to provide means for the temporary dilation of a blood vessel. Nearly all of the known devices described for this intended use are related to angioplasty and valvuloplasty balloon catheters. These inventions generally do not provide means to allow for blood flow during the time that the balloon is inflated and dilation is taking place.

A non-balloon intravascular dilation device that permits blood flow during vessel dilation is described in U.S. Pat. No. 5,653,684 (Laptewicz et al.). This invention incorporates a flexible wire mesh catheter tip that is used to compress flow obstructing material against the interior wall of a vessel and thereby return the diameter of the vessel to a sufficient diameter to allow normal flow in the vessel. This device is intended to remain in the vessel for periods of up to 48 hours. It is not designed for substantially expanding the diameter of a vessel for the purpose of reducing outflow resistance.

Prior art devices use expandable wire mesh structures to expand the lumen of a generally tubular body structure. Examples of these devices are provided in U.S. Pat. No. 4,347,846 (Dormia) and U.S. Pat. No. 4,590,938 (Segura et al.). These devices are useful primarily for the retrieval of obstructions such as stones from non-vascular ducts. The basket that is expandably formed from the wire mesh is geometrically asymmetrical in some respect to allow for both the capture and retention of the obstructive stone. The devices incidentally dilate the body structure when they are expanded to capture the obstruction, but the devices are not designed for use in dilating blood vessels and do not remain in the body for longer than is required for the retrieval procedure.

### SUMMARY OF THE INVENTION

An object of this invention is to provide improved devices and improved treatment methods to effect many of the same therapeutic support functions as current mechanical and electromechanical therapies for acute heart failure, whereby the improved devices and related treatment methods also are significantly less complex than those of the known prior art. The present treatment for one embodiment of the invention, involves percutaneous transluminal introduction and positioning of a temporary one-way valve in series with the patient's essentially normal natural aortic valve. The valve may be positioned in the ascending aorta near the natural aortic valve, at the beginning of the descending aorta or at a site in between these two positions. The valve is actuated (opened) by the expulsion of blood from the heart, in the same way that the natural aortic valve is opened. The temporary one-way valve of this invention requires no external power source or timing mechanism. The valve closes at the end of systole and relieves much of the systemic back-pressure that affects the natural valve and the left ventricle and thereby improves the performance of the left ventricle. This improvement in performance may be noted by an improvement (increase) in cardiac output and ejection fraction, and a decrease in heart rate and pulmonary capillary wedge pressure. These changes tend to decrease myocardial oxygen demand and thus allow the heart to recover from the episode of acute ventricular failure. The present treatment for a second embodiment of the invention involves percutaneous transluminal placement of a temporary dilatation means in the descending aorta to increase the diameter (and thus the volume) of that portion of the outflow path engaged by the device and thereby decreases the outflow resistance. The valve component and the dilation component of the first and second embodiments may be used alone or together in a given patient.

The one-way valve assembly embodiment consists of an annulus, a frame or annulus support structure, valve leaflets, and control means to both advance the collapsed valve through the arterial tree to the site of deployment and later to remove the valve, control means to deploy the valve, and a structure to prevent prolapse of the leaflets in some configurations of the valve.

The temporary vessel dilatation device consists of an expansible frame that may be percutaneously transluminally introduced in a collapsed form from an access site in a peripheral artery, such as the femoral artery. In a preferred embodiment, the temporary dilation device takes the form of a cylindrical cage that can be expanded after being positioned at the desired site to enlarge the diameter of an associated lumen portion of the descending aorta while allowing blood to flow freely through its natural course.

The present inventive devices, as indicated, include a collapsible valve and a vascular dilation device that are introduced through percutaneous transluminal techniques either as part of a cooperating system or separately. Use of these devices and the disclosed treatment method offers temporary support to the injured heart to allow recovery without the need for a substantially more complex system involving powered pumping and timing mechanisms.

### BRIEF DESCRIPTION OF THE DRAWINGS

Various embodiments of the present invention are described in detail below with reference to the drawings, in which like items are identified by the same reference designation, wherein:

FIG. 1a is a perspective view of the distal end of one embodiment of the invention for a temporary valve assembly illustrating the frame in the deployed position with the valve partially open within a cross sectional portion of an aorta;

FIG. 1b is a top view from the distal end of the temporary valve assembly absent the frame below the annulus of FIG. 1a;

FIG. 2a is a perspective view of the distal end of an alternative embodiment of the invention for a temporary valve assembly illustrating the frame in the deployed position with the valve partially open within a cross sectional portion of an aorta;

FIG. 2b is a top view from the distal end of the temporary valve assembly absent the frame below the annulus of FIG. 2a;

FIG. 3 is a perspective view of the distal end of an alternative embodiment of the invention for a temporary valve assembly illustrating the frame in the deployed position with the valve partially open within a cross sectional portion of the aorta;

FIG. 4 is a perspective view of the distal end of an alternative embodiment of the invention for a temporary valve assembly illustrating the frame in the deployed position with the valve partially open within a cross sectional portion of the aorta;

FIG. 5a is a perspective view of an alternative embodiment of the temporary valve assembly shown in FIG. 2a illustrating the frame in the deployed position with the valve partially open within a cross sectional portion of the aorta;

FIG. 5b is a perspective view of an alternative embodiment of the temporary valve assembly shown in FIG. 4 illustrating the frame in the deployed position with the valve partially open within a cross sectional portion of the aorta;

FIG. 5c is an enlarged view of a portion of FIGS. 5a and 5b;

FIG. 6 is a side view of the valve assembly of FIG. 1a inserted in the aorta in one position consistent with the treatment method of the invention;

FIG. 7 is a side view of the valve assembly of the present invention inserted in the aorta in an alternate position relative to that of FIG. 6 consistent with the treatment method of the invention;

FIG. 8 is a side view of one embodiment of the invention showing the vascular dilation device in an expanded state within a cutaway portion of the descending aorta independently of the valve assembly catheter;

FIG. 9a is a side view of an alternative embodiment of the invention for a vascular dilation device in a partially expanded state and concentrically disposed about a valve assembly catheter within a cutaway portion of the descended aorta;

FIG. 9b is a side view of the vascular dilation device, as illustrated in FIG. 9a, but in a fully expanded position; and

FIG. 10 is of a partial cross sectional view of a valve assembly and a dilatation device of embodiments of the present invention simultaneously deployed in a cutaway portion of the aorta for a treatment method embodiment of the present invention.

### DETAILED DESCRIPTION OF THE INVENTION

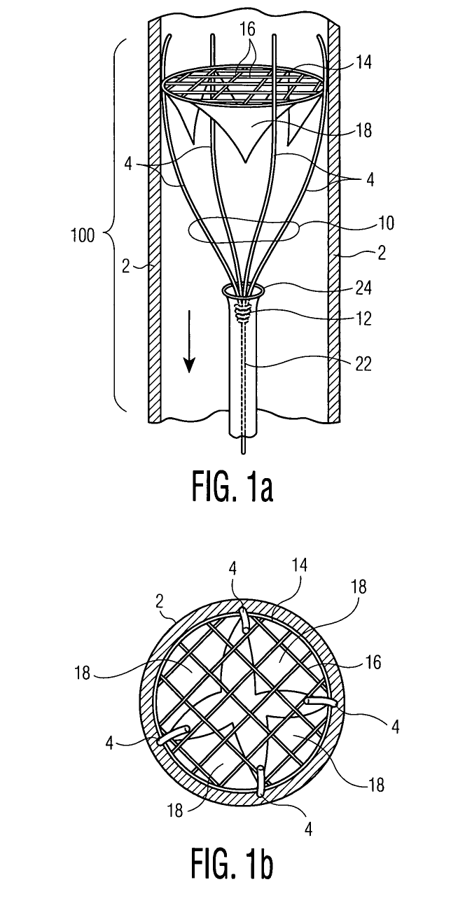

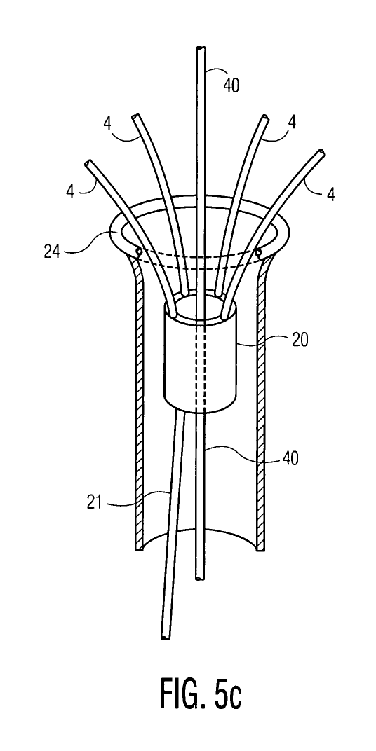

In general, the components of a collapsible valve assembly 100 as inserted in a thoracic aorta 2 are illustrated in FIG. 1a, for one embodiment of the invention. It should be noted that not all of the component elements shown are required for each exemplary embodiment illustrated herein. Examples of such possible variations will be described with reference to the various drawings. The components shown include an expandable frame 10 comprising a plurality of radially outwardly biased wires 4 or bands joined together at least at one end 12 to form a cage-like structure that may be open at the end opposite the joining point. Still other frame structures are discussed with respect to other of the present drawings. The collapsed diameter dimension of the valve assembly 100 is between 2 mm and 5 mm, and the expanded diameter dimension of the frame 10 suitable for application in an adult patient is between 20 mm and 35 mm. The wires 4 may be joined by welding or other adhesive means to a hollow cylinder 20 (see FIG. 5c) or directly to each other at the apex 12 of the conic or bulbous frame 10 and to an elongated control element 22 that extends out of the body through the remote percutaneous access site (not shown). The control element 22 may be a wire or a flexible tube that must possess adequate column strength so as to allow the valve assembly 100 to be deployed from the confinement of a guide catheter 24, (such as the type typically used in vascular access procedures), and adequate tensile strength to allow safe withdrawal of the valve assembly 100 into the guide catheter 24 prior to removal from the body. Upon introduction of the valve assembly 100 into the thoracic aorta 2 the distal end of the guide catheter 24 is positioned at the intended deployment site. The catheter 24 is then retracted while maintaining counter tension on the control element 22. The radially outwardly biased frame structure 10 is thus allowed to expand so as to cause the greatest diameter of the frame to frictionally engage the interior wall of the thoracic aorta 2. The wires 4 or bands that form the expandable frame 10 can be of preformed, outwardly biased spring construction, or can be fabricated of shape memory material such as nitinol, or can be radially expandable by means of a control element (as discussed later in this description). The expandable valve frame 10 of the valve assembly 100 embodiment shown in FIG. 1a forms a generally conic or bulbous shape when expanded. The individual frame wires 4 or bands of the valve frame 10 are connected to a valve annulus 14 in the form of a flexible strand at the point that forms the greatest diameter of the expanded frame 10. When ideally deployed and functioning the plane formed by the annulus 14 is maintained at right angles to the direction of blood flow during systole (see arrow). The annulus 14 can be disposed within or outside of the frame members 10 in a generally circular plane. The annulus 14 may be covered with flexible polymeric material so as to form a seal at its interface with the wall of the aorta. The free ends of the frame wires 4 can terminate at the plane of the annulus 14 or extend beyond the plane of the annulus to provide additional surface contact area with the wall of the thoracic aorta 2. The multiple valve leaflets 18 (at least 3) are attached to the annulus 14 so as to form a one-way valve that permits a central flow of blood during cardiac systole (see direction of arrow) and to prevent or minimize back flow during diastole. The leaflets 18 are preferable formed of a thin, flexible, clot resistant, biocompatible polymeric material such as polyester or polyurethane and are attached to the annulus by suturing, adhesives or other suitable means. The leaflets 18 either form a conic shape with the apex of the cone located distal to (downstream on the annulus 14, when the one-way valve is in the closed position, or close in a plane described by the annulus 14. When the leaflets 18 are arranged so as to close in a plane they seat against a prolapse prevention element 16. This component of the valve assembly 100 lays in the plane of the annulus 14 immediately proximal (upstream of) the valve leaflets 18 and is formed by at least four filaments that form a grid within the plane of the circle formed by the annulus 14, for example. In a preferred form the prolapse prevention component 16 is a metallic or polymeric mesh disc with the open area of the grid accounting for at least 70% of the total area described by the annular space. The valve leaflets 18 may be attached directly to the periphery of the prolapse prevention element 16 rather than to a physical annulus. In this case the periphery of the prolapse prevention element 16 serves as a “virtual” annulus and no separate annular ring is required in the valve assembly 100. In such an embodiment, the periphery of the prolapse prevention element 16 is reinforced with flexible polymeric material so as to form a seal at its interface with the wall of the thoracic aorta 2.

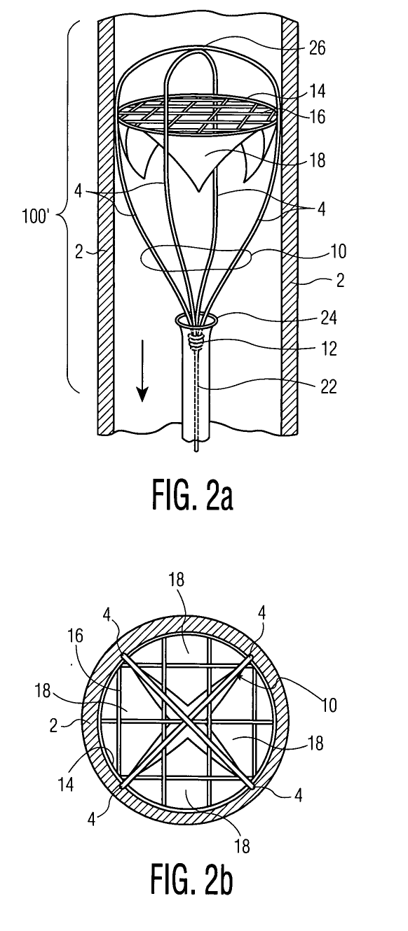

The embodiment of the valve assembly depicted in FIG. 2a differs from the valve assembly shown in FIG. 1a with respect to the construction of the valve frame 10. This alternate valve frame assembly 100′ also assumes a generally conic or bulbous shape upon expansion. However, the individual wires or bands of the valve frame are not only joined at the apex 12 of the cone, but are either continually extended to pass through a point opposite the apex 26 or joined at a point opposite the apex 26 to enclose the annulus plane and thus form a closed bulb shaped cage. This construction adds stiffness to the frame structure, provides increased stability by increasing the area of frame contact with the wall of the thoracic aorta 2, and provides increased assurance that the plane of the valve annulus 14 remains at right angles to the direction of blood flow during systole (see arrow).

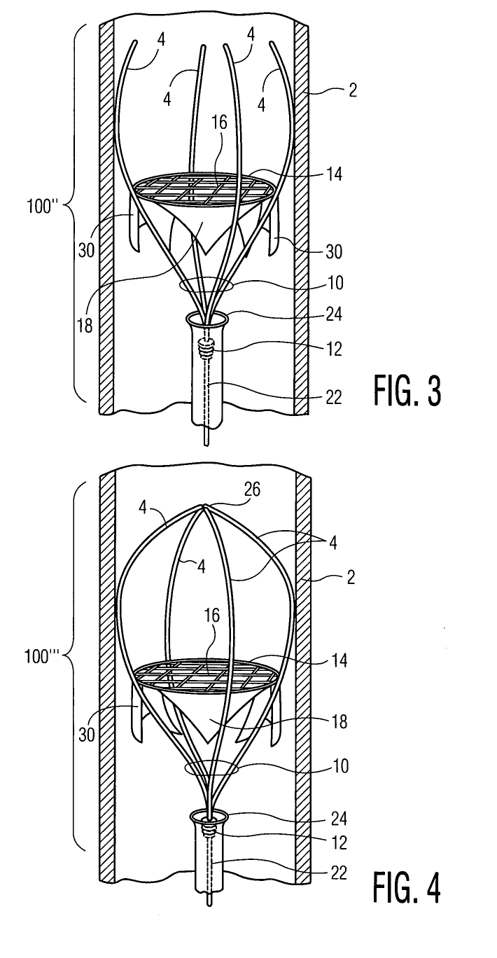

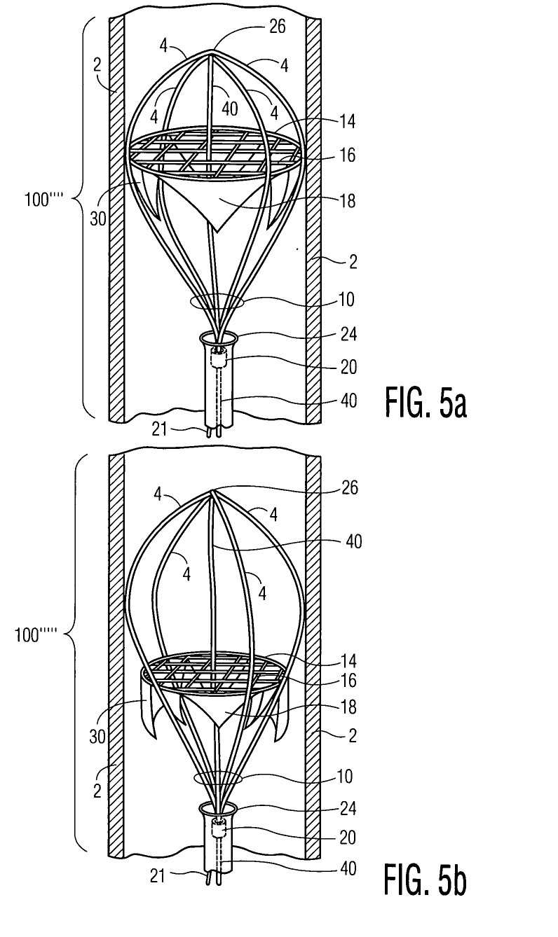

The valve assembly embodiments illustrated in FIG. 1a and FIG. 2a must be sized for specific aorta diameter dimensions. The thoracic aorta 2 of an adult human ranges in diameter from approximately 19 mm to 31 mm in over 90% of the population. Typical replacement valves used to supplant a diseased non-functional aortic valve are made available in 2 mm increments over this diameter range. FIG. 3 and FIG. 4 depict embodiments of the valve assemblies 100 and 100′ that are configured to allow a single valve assembly size to be used over most or all of the range of adult aorta diameters. This is accomplished by modifying the location of the plane of the annulus 14 and adding a secondary set of leaflets 30, for example. The valve assembly 100″ of FIG. 3 is otherwise analogous in design to the valve assembly 100 depicted in FIG. 1a; and the valve assembly 100′″ of FIG. 4 is otherwise analogous in design to the valve assembly 100′ shown in FIG. 2a. The annulus 14 planes of the valve assemblies 100″ and 100′″ shown in FIG. 3 and FIG. 4, respectively, have been shifted toward the apex 12 of the frame assembly. In these embodiments the annulus 14 plane is at a point where the diameter described by the members of the frame assemblies 100″ and 100′″ is typically between 20 mm and 24 mm so that the annulus diameter occupies at least 50% of the aorta diameter. In order to prevent any significant retrograde blood flow during diastole, the periphery of the annulus 14 is fitted with at least three thin, flexible, biocompatible leaflets 30 that are attached at their fixed edges to the annulus 14 or the prolapse prevention element 16 to form a skirt. The leaflets 30 are generally trapezoidal in shape with the lesser length attached to the annulus 14. These leaflets 30 operate in concert with the central leaflets 18 to open and permit antegrade blood flow during systole and close to prevent retrograde blood flow during diastole. The free edges of the peripheral leaflets 30 engage the wall of the thoracic aorta 2 during diastole to prevent any substantial retrograde flow.

The embodiments depicted in FIG. 1 through FIG. 4 share the characteristic feature that expansion of the valve assembly is accomplished through the action of the radially outwardly biased wire or band members 4 of the frame 10. The alternative expandable valve assemblies 100″″ and 100′″″ illustrated in FIG. 5a and FIG. 5b, respectively, differ from the valve assembly embodiments 100, 100′, 100″, and 100′″ depicted and described previously in this description with respect to the means for expanding the valve frame from its collapsed configuration to its expanded, deployed configuration. The valve assembly 100″″ depicted in FIG. 5a is analogous to the valve assembly 100′ shown in FIG. 2a, and the valve assembly 100′″″ depicted in FIG. 5b is analogous to the valve assembly 100′″ shown in FIG. 4 with regard to the respective locations of the annulus 14 planes. In both FIG. 5a and FIG. 5b the wire or band members 4 of the valve frame 10 are joined at a point or apex 26 at one end, and at their opposite ends to one end of a hollow cylinder 20 (see FIG. 5c). There is additionally attached a control member 40 that extends from the point or apex 26 through the longitudinal axis of the respective valve assembly 100″″, 100′″″, through the hollow cylinder 20, and thence through the central channel of the flexible catheter 24 to a point outside of the body where it is connected to an actuation means. The flexible wire or band members 4 of the valve frame 10 depicted in FIG. 5a and FIG. 5b are not sufficiently radially outwardly biased to cause deployment of the respective valve assembly 100″″, 100′″″ upon advancement of the valve assembly from the confinement of the catheter 24 by action of the control wire 21 attached to the cylinder 20 of the respective valve assembly 100″″, 100′″″. Instead, the alternative valve assemblies 100″″, 100′″″ of FIG. 5a and FIG. 5b are deployed by positioning the distal end of the catheter 24 at the desired site, retracting the catheter 24 while maintaining the position of the control wire 21, followed by retraction of the central control member 40. This combination of actions by the operator releases the respective valve assembly 100″″, 100′″″ from the confinement of the catheter 24 and then compresses the frame longitudinally to expand the diameter and complete deployment of the valve assembly. In the case of these alternative configurations, the control wire 40 passes through a central point in the plane of the valve annulus 14 without interfering with the functional operation of the valve leaflets 18 and/or 30.

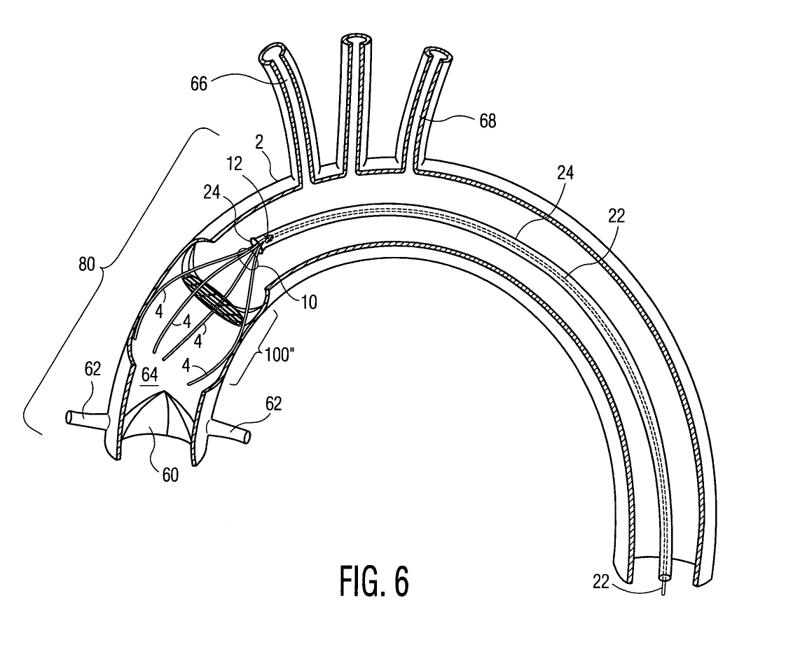

FIG. 6 is a generalized overview of one embodiment of the collapsible valve assembly 100″ shown in its expanded, deployed configuration within the ascending aorta 80 during cardiac diastole. In this preferred position the valve assembly is placed at a site between the natural aortic valve 60 and the brachlocephalic trunk 66, the first major arterial branch of the aorta. There is an adequate space 64 and thus, sufficient intraluminal volume to allow normal flow of blood to the coronary arteries 62 during cardiac diastole. In this position the temporary valve assembly 100″ bears a great proportion of the systemic blood pressure during diastole; and thus the back-pressure on the natural aortic valve 60 is largely relieved. Upon contraction of the left ventricle and opening of the aortic valve 60 outflow resistance is reduced relative to the situation where the temporary valve 100″ is not deployed.

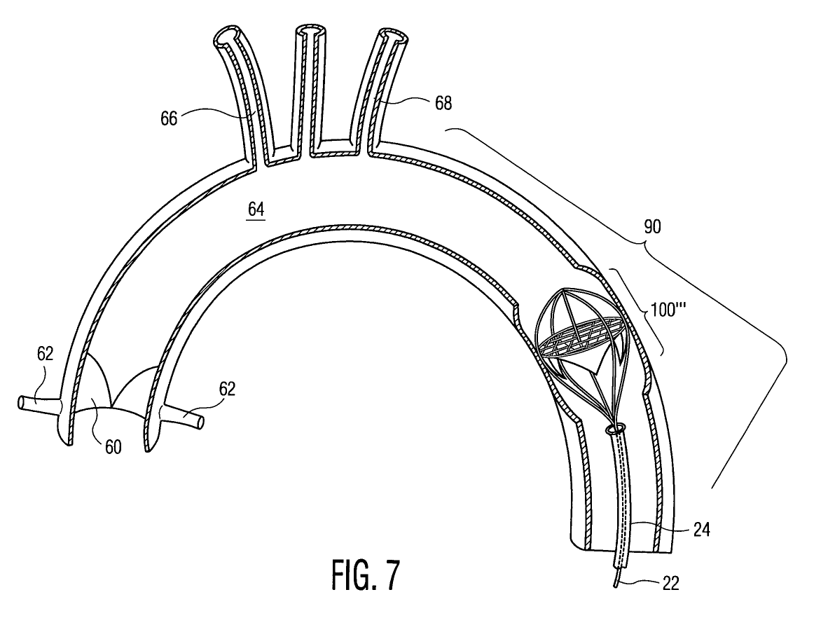

The valve assembly overview illustrated in FIG. 7 shows one embodiment of the collapsible valve assembly 100′″ in its expanded, deployed configuration within the descending thoracic aorta 90 during cardiac systole. In this position, the valve assembly is placed distal to the left subclavian artery 68, the third major arterial branch of the aorta. When positioned at this alternative site or at locations in between this site and the location depicted in FIG. 6 for a valve assembly 100″, the temporary valve 100′″ will also bear a portion of the systemic blood pressure during cardiac diastole and thus relieve a portion of the back-pressure on the natural aortic valve 60. By relocating the valve assembly 100′″ from the position of the valve assembly 100″ shown in FIG. 6, toward the position depicted in FIG. 7, it is possible to gradually wean the patient from temporary support of cardiac function. If, upon such repositioning, cardiac performance is not acceptable, as determined by such means as electrocardiographic and hemodynamic measurements, the temporary valve assembly 100″ or 100′″ may be again repositioned at a point nearer the natural aortic valve 60 for an additional period of time. Once satisfied with cardiac performance, the operator can undeploy the temporary valve 100″ or 100′″ into the catheter 24, and withdraw the catheter and valve assembly 100″ or 100′″ as a unit from the body.

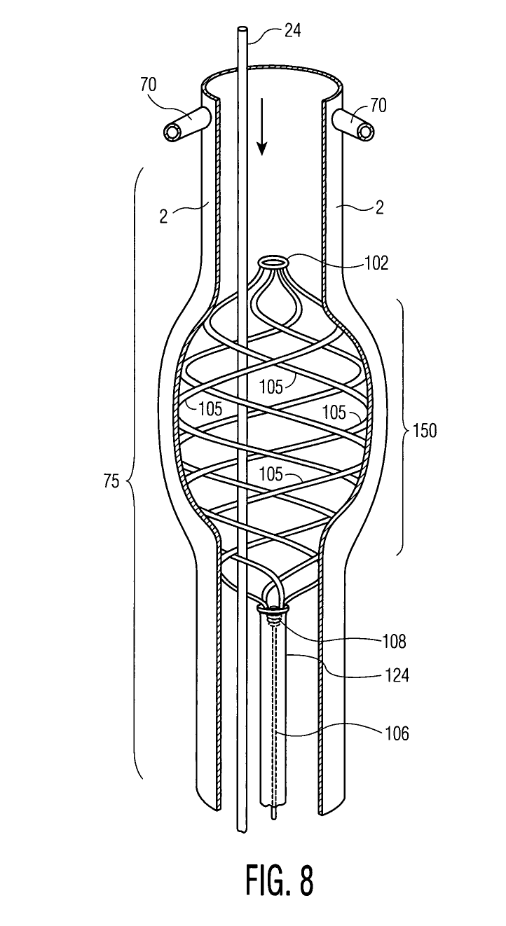

FIG. 8 is a side view of one embodiment of a temporary vascular dilation device assembly 150 of the present invention positioned in the intrarenal abdominal aorta 75 with the dilation device shown in the expanded state. The dilation device assembly 150 is preferably deployed in the intrarenal abdominal aorta (distal to the renal arteries 70), but alternatively may be deployed in a more distal portion of the arterial system such as in the iliac or femoral arteries. When the device is deployed the volume of the arterial system may be increased by up to 200 cc, thus decreasing outflow resistance and encouraging an improvement in cardiac output and left ventricular ejection fraction. The temporary vascular dilation device depicted in FIG. 8 is designed for introduction into the body independently of the temporary valve assembly of this invention. The dilation device may be percutaneously introduced and deployed prior to insertion of the valve assembly catheter 24, which can be subsequently inserted through the openings in the expandable dilation device assembly 150.

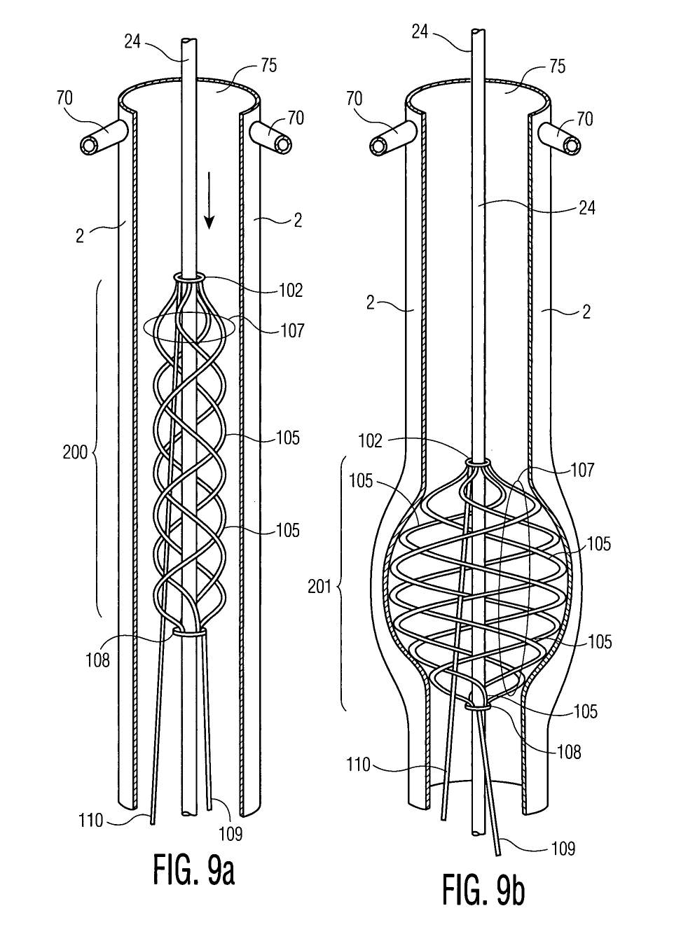

Several alternate configurations 150, 200, and 201 of the dilation device assembly are described below with reference to the respective drawings. It should be noted that not all of the component elements shown are required for each exemplary embodiment illustrated herein. Examples of such possible variations will be described with reference to the various drawings. The components shown include a self-expandable frame 150 comprising a plurality of radially outwardly biased wires or bands 105 in the embodiment of FIG. 8 joined together at top end 102, and at bottom end 108 to form a generally cylindrical symmetrical cage-like structure 150. The collapsed diameter dimension of the vascular dilation assembly is ideally between 1 mm and 6 mm and the expanded diameter dimension of the dilation assembly suitable for application in an adult patient is between 25 mm and 50 mm. The wires 105 can be joined together by swaging, welding or other connecting means to a cylindrical ring or directly to each other at each end 102 and 108 of the generally cylindrical cage, for example. The wires or bands 105 that form this cage can be disposed parallel to each other, or alternately disposed in a clockwise/counter clockwise helical fashion or may be formed into a braided structure. The proximal end 108 of the cylindrical cage 150 of FIG. 8 is connected to an elongated control element 106 that extends through a dedicated guide catheter 124 and thence out of the body through the remote percutaneous access site (not shown). The control element 106 can be a wire or a flexible tube with adequate column strength so as to allow the dilation device to be deployed from the confinement of a guide catheter 124, (such as the type typically used in vascular access procedures), and adequate tensile strength to allow safe withdrawal of the dilation device into the guide catheter 124 prior to removal from the body, for example. Upon introduction of the dilation device into the abdominal aorta the distal end of the guide catheter 124 is positioned at the intended deployment site. The catheter 124 is then retracted while maintaining counter tension on the control element 106. The radially outwardly biased cylindrical cage structure 150 is thus allowed to expand so as to cause the expanded diameter of the cage structure 150 to frictionally engage and dilate the wall of the intrarenal abdominal aorta 75. The wires or bands 105 that form the self-expandable frame 150 can be of preformed, outwardly biased spring construction, and/or fabricated of shape memory material such as nitinol. The embodiments of FIGS. 9a and 9b are radially expandable by means of control elements (as discussed below), for example.

The partially deployed temporary dilation assembly 200 shown in FIG. 9a is slidably mounted concentrically on the guide catheter 24 of the temporary valve assembly 100, or 100′, or 100″, or 100′″, or 100″″. The guide catheter 24 passes through cylindrical rings 102 and 108 at each end of an expandable frame 107. After the temporary valve assembly 100, or 100′, or 100″, or 100′″, or 100″″ is positioned at the desired location and deployed, the temporary dilation assembly 200 may be positioned at its preferred location by advancing a control element 109 that is attached to either one of the slidable rings 102 or 108, at an end of the expandable frame 107. In this example, the cage frame 107 can be expanded to its deployed position by applying opposing forces on two control elements, 109 and 110, attached respectively to the rings, 108 and 102, at the proximal and distal ends of the cage assembly 107. For example, the cage 107 is expanded by applying a retraction force to control element 110 while holding control element 109 in a fixed position, thereby dilating the engaged section of the intrarenal abdominal aorta 75.

In another embodiment of the invention, the proximal end (ring 108) of a fully deployed dilation assembly 201 depicted on FIG. 9b is fixedly mounted to the guide catheter 24. In the case where the proximal ring 108 of the dilation assembly 201 is fixed to the guide catheter 24, the expandable frame 107 is expanded by applying tension (retraction force) to the control element 110 attached to the slidable end 102 of the dilation assembly 201. In an alternative case, the fixed end and the slidable end are reversed, the cage can then be expanded by fixing the distal end (ring 102) of the dilation assembly to the guide catheter 24, and applying compressive force to (advancing) the control element 109 attached to the slidable (proximal) end 108 of the dilation assembly 201.

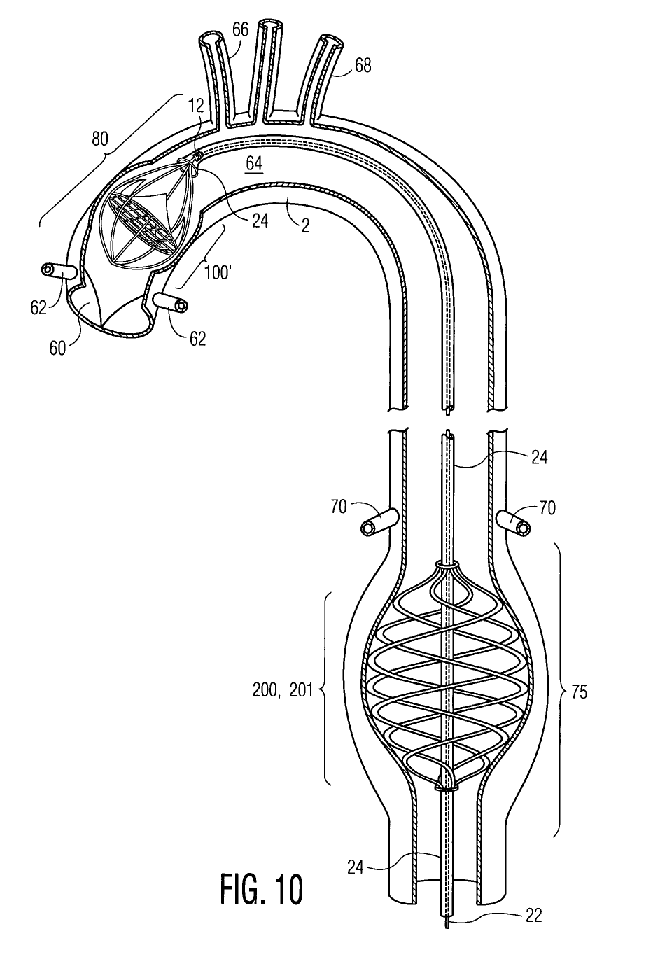

FIG. 10 is an overview showing the in vivo placement of the temporary valve assembly 100′ in position in the ascending aorta 80, and the temporary dilation assembly 200 in a dilated state positioned in the intrarenal aorta 75.

It is believed that the various embodiments of the invention described above may improve cardiac performance as measured by such criteria as any of: reduced outflow resistance, increased ejection fraction, increased cardiac output, decreased diastolic pressure on the natural aortic valve, decreased heart rate and/or decreased pulmonary capillary wedge pressure depending on the status and condition of a specific patient.

Although various embodiments of the invention have been shown and described, they are not meant to be limiting. Those of skill in the art may recognize certain modifications to these embodiments, which modifications are meant to be covered by the spirit and scope of the appended claims.

1. (canceled)

2. (canceled)

3. (canceled)

4. (canceled)

5. (canceled)

6. (canceled)

7. (canceled)

8. (canceled)

9. Apparatus for jointly providing intravascular treatment of acute left ventricular heart failure comprising:
a) a collapsible temporary valve assembly including means for deploying it into the aorta via its introduction into the vascular system using percutaneous transluminal techniques, and means for both expanding and deploying it in the aorta; and
b) a collapsible vessel dilation assembly including means for deploying it into the aorta or peripheral arteries via its introduction into the vascular system using percutaneous transluminal techniques, and means for both expanding its diameter and deploying it in the aorta or peripheral arteries.

10. A device for the intravascular treatment of acute left ventricular heart failure comprising:
a collapsible temporary valve assembly including means for deploying it into the aorta via its introduction into the vascular system using percutaneous transluminal techniques, and means for both expanding and deploying it in the aorta.

11. A device for the intravascular treatment of acute left ventricular heart failure comprising:
a collapsible vessel dilation assembly including means for deploying it into the aorta or peripheral arteries via its introduction into the vascular system using percutaneous transluminal techniques, and means for both expanding its diameter and deploying it in the aorta or peripheral arteries.

12. A collapsible temporary valve assembly for deployment into the aorta via introduction into the vascular system using percutaneous transluminal techniques comprising:
a collapsible frame having at least three wires or bands joined at least at one end and biased radially outward so as to form a generally conic or bulbous cage upon deployment;
a control element joined directly or indirectly to the collapsible frame members at their juncture at the apex of the frame and extending to a point outside of the body to allow expansion and collapse of the frame by alternately allowing advancement of the frame from a constraining catheter and retraction of the frame into the catheter, whereby upon expansion of the frame the valve assembly functions in series with a patient's essential normal aortic valve, thereby allowing the temporary valve assembly to decrease the back pressure on the natural valve when both are closing during the diastolic phase of the cardiac cycle;
an annulus in the form of a flexible strand disposed within or around the frame in a plane perpendicular to the longitudinal axis of the frame in the deployed position or a fluid permeable mesh disc disposed within the frame in a similar plane; and
at least three thin, flexible, biocompatible leaflets attached at their fixed edges to the annular strand or the mesh disc and configured to permit central flow of blood during cardiac systole and to substantially prevent retrograde flow of blood during cardiac diastole.

13. The valve assembly of claim 12, further including:
remote sensing means for capturing physiological data including intra arterial pressure, cardiac output, pulse rate, and other desired data.
